(19) United States
(12) Patent Application Publication
(10) Pub. No.: US 2021/0322596 A1
(43) Pub. Date: Oct. 21, 2021

(54) ULTRAVIOLET SANITIZATION FIXTURE

(71) Applicant: Magtech Industries Corporation, Las Vegas, NV (US)

(72) Inventors: Michael Kuo, Las Vegas, NV (US); James Highgate, Las Vegas, NV (US)

(73) Assignee: Magtech Industries Corporation, Las Vegas, NV (US)

(21) Appl. No.: 17/230,936

(22) Filed: Apr. 14, 2021

Related U.S. Application Data
(60) Provisional application No. 63/012,656, filed on Apr. 20, 2020.

(51) Int. Cl. A61L 2/10

(52) U.S. Cl. CPC A61L 2/10; A61L 2202/25; A61L 2202/14; A61L 2202/11

(57) ABSTRACT

An ultraviolet sanitization fixture comprising a plurality of emitters that emit UVC radiation to render various pathogens inert. The plurality of emitters may be organized into a number of sets, with each set emitting distinct UVC radiation wavelengths and being independently controlled. One of the sets of emitters may be operated according to various emission thresholds and reset triggers. In this manner, the ultraviolet sanitization fixture efficiently provides germicidal effects while taking health and safety into account.

### CROSS-REFERENCE TO RELATED APPLICATION

This application claims priority to U.S. Provisional Patent Application No. 63/012,656, filed Apr. 20, 2020.

### BACKGROUND OF THE INVENTION

### Field of the Invention

The invention relates to lighting devices and in particular to an ultraviolet sanitization fixture.

### Related Art

Electromagnetic radiation within the ultraviolet (“UV”) spectrum is known to have germicidal properties. As such, electromagnetic emissions can be used for sanitization purposes. For instance, a log reduction in microbial life has been shown due to UV exposure.

The UV spectrum extends between the visible light and X-ray spectrums. Within this range, three categories of UV radiation exist. These are, from longest to shortest wavelengths, UVA, UVB, and UVC. More specifically, UVA, UVB, and UVC can be generally thought of as the 320-400 nm, 290-320 nm, and 100-290 nm wavelength ranges, respectively speaking.

From the discussion that follows, it will become apparent that the present invention addresses the deficiencies associated with the prior art while providing numerous additional advantages and benefits not contemplated or possible with prior art constructions.

### SUMMARY OF THE INVENTION

An ultraviolet sanitization fixture is disclosed herein. The ultraviolet sanitization fixture sanitizes an area through UV radiation, typically, UVC radiation emitter from one or more emitters thereof. The ultraviolet sanitization fixture may independently control various sets of emitters to efficiently provide a germicidal effect while accounting for human health and safety relative to UVC radiation.

Various systems and methods related to an ultraviolet sanitization fixture are disclosed herein. For example, in one embodiment an ultraviolet sanitization fixture comprises one or more primary emitters that emit UVC radiation within a first wavelength range, one or more secondary emitters that emit UVC radiation with a distinct second wavelength range, and a controller. The first wavelength range may be between 207 nm and 222 nm and the second wavelength range may be between 254 nm and 265 nm.

The controller records emission activity of the secondary emitters, activates or deactivates the secondary emitters based on one or more thresholds relative to the recorded emission activity, and resets the recorded emission activity based on one or more predefined reset triggers.

The emission activity may comprise emission time or power output. The predefined reset triggers may comprise a time limit or detection of a human presence. One or more sensors may be included as well. The controller may deactivate the secondary emitters when a human presence is detected by the sensors in these embodiments.

In another exemplary embodiment of an ultraviolet sanitization fixture the controller records emission activity of the secondary emitters, and activates or deactivates the secondary emitters based on one or more limits relative to the recorded emission activity. The primary emitters are activated regardless of whether the secondary emitters are activated or deactivated.

The controller may reset the recorded emission activity based on an elapsed time. The controller may also reset the recorded emission activity based on detection of a human presence by one or more sensors or based on one or more temperature thresholds measured by one or more sensors. The emission activity may comprise emission time or power output. The primary emitters may be activated on a substantially constant basis.

Various methods relating to the ultraviolet sanitization are disclosed herein as well. In one exemplary method, a method of ultraviolet sanitization with an ultraviolet sanitization fixture comprising one or more primary emitters and one or more secondary emitters is disclosed.

The method comprises activating the primary emitters, determining emission activity of the secondary emitters with a controller starting from an occurrence of one or more triggering events, and activating or deactivating the secondary emitters based on one or more limits relative to the emission activity.

The primary emitters emit UVC radiation within a first wavelength range and the secondary emitters emit UVC radiation with a second wavelength range distinct from the first wavelength range. The primary emitters are activated regardless of whether the secondary emitters are activated or deactivated.

The triggering events may comprise a predefined period of time, detection of a human presence by one or more sensors, or detection of one or more particular temperatures by one or more sensors. The emission activity may comprise emission time or power output.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 is a side view of an exemplary ultraviolet sanitization fixture;

FIG. 2 is a bottom view of an exemplary ultraviolet sanitization fixture;

FIG. 3 is a flow diagram illustrating operation of an exemplary ultraviolet sanitization fixture;

FIG. 4 is a flow diagram illustrating operation of an exemplary ultraviolet sanitization fixture; and

FIG. 5 is a block diagram illustrating an exemplary controller.

### DETAILED DESCRIPTION OF THE INVENTION

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

Generally speaking, the ultraviolet sanitization fixture herein emits invisible ultraviolet (“UV”) radiation that has a germicidal effect. The germicidal effect may inactivate or destroy a variety of pathogens, including bacteria, viruses, and fungi. As such, the ultraviolet sanitization fixture may be used for sanitization purposes. For instance, the ultraviolet sanitization fixture may irradiate sanitization targets such as various surfaces, the air, or both to sanitize the same. The pathogens that are present are thereby rendered inert to a substantial or a particular degree.

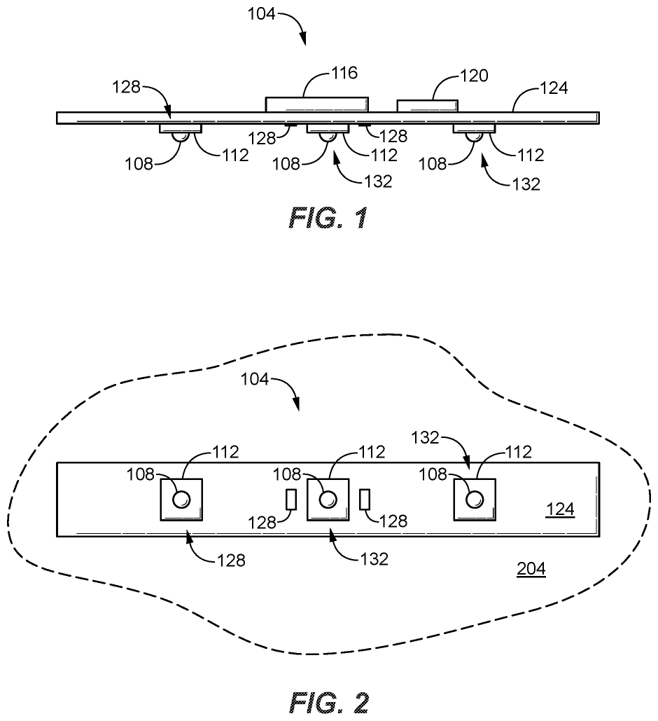

FIG. 1 provides a side view of an exemplary ultraviolet sanitization fixture 104. As can be seen, an ultraviolet sanitization fixture 104 may comprise one or more emitters 112, one or more sensors 128, and one or more controllers 116.

An emitter 112 may emit UV radiation via one or more emitter elements 108. Typically, an emitter element 108 will be an LED; however, it is contemplated that other elements capable of emitting UV radiation may be used.

Typically, invisible UVC radiation will be emitted by an ultraviolet sanitization fixture, such as via particular emitter elements 108 suited to do the same. UVC radiation is absorbed by RNA and DNA as well as by proteins of pathogens, rendering the same inert through inactivation or death.

As can be seen, a plurality of emitters 112 may be provided. The emitters 112 may emit distinct wavelengths of UVC radiation. Various wavelengths of UVC radiation may accordingly be emitted at and for various times during operation of an ultraviolet sanitization fixture. This allows a germicidal effect to be safely provided, even in offices, medical facilities, residences, or other areas that may have a human presence.

In one or more embodiments, the emitters 112 of an ultraviolet sanitization fixture 104 may be divided into at least two distinct sets. For example, there may be one or more primary emitters 128 and one or more distinct secondary emitters 132. The distinct sets of emitters 112 may each emit distinct wavelengths of UVC radiation.

For instance, one or more primary emitters 128 may emit UVC radiation between 207 nm and 222 nm, while one or more secondary emitters 132 may emit UVC radiation between 254 nm and 265 nm. These wavelength ranges have been selected for their germicidal effect as well as safety characteristics. UVC radiation between 207 nm and 222 nm has a high germicidal effect while not penetrating the skin or eyes to a sufficient extent to cause harm. UVC radiation between 254 nm and 265 nm similarly has minimal harmful effects; however, the ultraviolet sanitization fixture 104 controls exposure to further minimize or eliminate harmful effects form these higher wavelengths of UVC radiation.

Together, these features allow a primary emitter 128 to continue to emit UVC radiation regardless of whether humans are present, while exposure to UVC radiation from a secondary emitter 132 can be controlled. This allows various health and safety guidelines to be met, such as ICNIRP and OSHA guidelines on UVC exposure.

In general, lower wavelengths of UVC radiation provide a higher germicidal effect over the same period of time relative to higher wavelengths. Shorter time periods are advantageous in that pathogens are not afforded time to mutate or become resistant. In addition, power consumption may be reduced when emitting lower wavelengths of UVC radiation.

Different wavelengths produce germicidal effects differently which can be used to increase effectiveness relative to a variety of pathogens. For example, photochemical reactions other than DNA absorption of the same may be caused by UVC radiation of higher wavelengths as compared to lower wavelengths. In addition, different species of types of pathogens may be more or less susceptible to higher or lower wavelengths of UVC radiation.

For example, E. coli is 15% more efficiently inactivated at 265 nm as compared to a UVC peak of 254 nm, as described in chapter 2 of W. Kowalski, Ultraviolet Germicidal Irradiation Handbook, DOI 10.1007/978-3-642-01999-9_2, © Springer-Verlag Berlin Heidelberg 2009.

It is contemplated that various wavelengths may be emitted by primary, secondary, or other sets of emitters. As stated above, these wavelengths will typically be distinct and typically fall within the UVC wavelength range. Additional details regarding such emissions are provided below.

One or more sensors 128 may be provided to detect the presence of one or more humans. Typically, a sensor 128 will detect whether a human is within a vicinity subject to UVC irradiation from an ultraviolet sanitization fixture. This allows emission of UVC radiation to be controlled based on the presence or absence of humans. Some exemplary sensors 128 include heat or motion sensors. It is contemplated that a plurality of the same or distinct sensors 128 may be utilized.

As will be described further below, one or more controllers 116 may control operation of all or various subsets of an ultraviolet sanitization fixture's emitters 112 based on various operating parameters. Such operating parameters may include sensor information from one or more sensors 128 and timing information. Timing information may include current time and elapsed time.

One or more power sources 120 may be provided to power an ultraviolet sanitization fixture 104. A power source 120 may be a driver in some embodiments. A power source 120 may provide power intrinsically, such as via one or more internal batteries, may be connected to an external source of power, such as a municipal power utility, or both.

The components of an ultraviolet sanitization fixture 104 may be affixed to a body 124. A body 124 may comprise one or more support structures such as frames, enclosures, housings, or the like. A body 124 may be a rigid structure or a flexible structure that can be made to conform to various shapes.

Though shown as having a rectangular shape with three emitters 112, it is contemplated that an ultraviolet sanitization fixture 104 may be constructed in various shapes and sizes and with fewer or additional emitters in the various embodiments of the invention.

FIG. 2 illustrates a bottom view of an exemplary ultraviolet sanitization fixture 104 in an exemplary environment of use. As shown, the ultraviolet sanitization fixture 104 is mounted to a ceiling 204. In this manner, UVC radiation can be emitted downward to sanitize the air and surfaces below. In addition, one or more sensors 128 may readily detect the presence of humans at such vantage point.

An ultraviolet sanitization fixture 104 may be direct-mounted to a ceiling 204 or may be suspended therefrom. In addition, an ultraviolet sanitization fixture 104 may be mounted to walls, furniture, or other supporting structures.

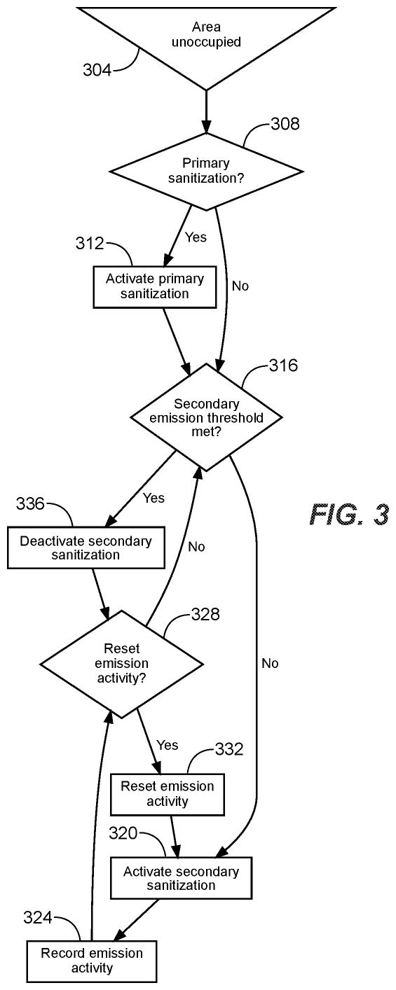

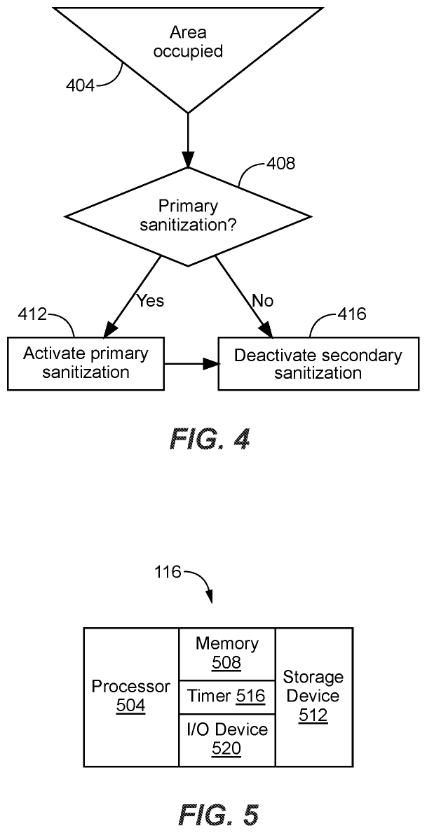

Operation of an exemplary ultraviolet sanitization fixture will now be described with respect to the flow diagrams of FIGS. 3 and 4. As can be seen, FIG. 3 illustrates operation of an exemplary ultraviolet sanitization fixture in an area unoccupied by one or more humans, while FIG. 4 illustrates operation of an exemplary ultraviolet sanitization fixture in an occupied area. As described above, the occupancy of an area may be determined by one or more sensors of an ultraviolet sanitization fixture.

As described above, an ultraviolet sanitization fixture's operation may be controlled by one or more controllers receiving operating parameters from one or more sensors, timing devices, or other source devices.

Referring to FIG. 3, at a step 304, it has been determined that the area is unoccupied. At a decision step 308 it may be determined whether primary sanitization 208 should be activated. Primary sanitization may be provided by UVC radiation from one or more primary emitters, as described above. Typically, primary sanitization will be constantly activated since UVC radiation from a primary emitter will not be harmful to humans. Accordingly, decision step 308 may not be provided in all embodiments of the ultraviolet sanitization fixture.

At a step 312, primary sanitization may be activated by activating one or more primary emitters to emit UVC radiation of their one or more particular wavelengths to provide a germicidal effect where irradiation occurs.

At a decision step 316, it is determined whether an emission threshold has been met, such as based on prior emission activity, as will be described further below. Typically, such threshold will apply to secondary sanitization effectuated by UVC radiation from one or more secondary emitters. For example, an emission threshold may be a time limit. This allows exposure and emission of the higher wavelength UVC radiation of the one or more secondary emitters to be controlled such that the UVC radiation is not emitted for longer than the predefined time limit. A time limit may be based on safety guidelines or rules for exposure time, germicidal effect, radiation wavelength, or various other factors.

If the emission threshold has not been met, secondary sanitization may be activated at a step 320 by activating one or more secondary emitters. As described above, secondary sanitization will typically provide one or more distinct germicidal effects due to the different wavelength or wavelengths of the one or more secondary emitters.

At a step 324, emission activity may be recorded. For example, emission activity may comprise timing information from a timing device, such as a clock or timer, that records the elapsed activation time of one or more secondary emitters. Emission activity may also include other information, such as power level. As stated, emission activity may be used in determining whether an emission threshold has been met at decision step 316.

At a decision step 328, it may be determined whether emission activity should be reset. By definition, emission activity does not meet an emission threshold upon reset. For example, timing information indicating the elapsed activation time of one or more secondary emitters may be reset to a predefined lower level, such as zero.

Emission activity may be reset on a periodic basis. For example, emission activity may be reset on a daily basis. In this manner, secondary sanitization is controlled accordingly to daily emission thresholds and emission activity. In the case of a time limit emission threshold, a daily time limit on secondary sanitization and its emission of UVC radiation is provided in this manner.

It is contemplated that a reset may occur on other basis as well. For example, emission activity may be reset manually or based on a triggering event, such as detection of pathogens or activity or conditions conducive to pathogen growth or presence as may be determined by one or more temperature, humidity, sound or other sensors. In one or more embodiments, a reset may be triggered by detection of a human to allow secondary sanitation to begin again once the area is unoccupied.

The following table, Table 1, provides some exemplary emission thresholds along with reset triggers for illustrative purposes. As can be seen, an emission threshold may be based on various types of thresholds, including time and power. In addition, reset triggers may be based on various timing or other events.

TABLE 1

| Radiation | Emission | | |
|---|---|---|---|
| Wavelength | Threshold | | Reset Trigger |
| 254-265 nm | 15 | Minutes | Daily |
| 254-265 nm | 15 | Minutes | Hourly |
| 266-290 nm | 10 | Minutes | Daily |
| 266-290 nm | 10 | Minutes | Hourly |
| 254-265 nm | 5 | Joules | Daily |
| 254-265 nm | 5 | Joules | Hourly |
| 266-290 nm | 5 | Joules | Daily |
| 266-290 nm | 5 | Joules | Hourly |
| 254-265 nm | 15 | Minutes | Human Presence Detection |
| 254-265 nm | 15 | Minutes | Temperature Threshold |
| | | | Detection |
| 254-265 nm | 15 | Minutes | Humidity Threshold Detection |
| 254-265 nm | 15 | Minutes | Sound Threshold Detection |

It is contemplated that a plurality of emission thresholds and reset triggers may be used simultaneously if desired. To illustrate, this would permit secondary sanitization to occur, for example, on a 15-minute threshold on a daily basis, but be repeated should a human or other trigger be detected.

A reset of emission activity may occur at a step 332. Thereafter, secondary sanitization may be activated again at step 320. If emission activity is not reset at decision step 328, it may be determined whether current emission activity meets the emission threshold at decision step 316. When an emission threshold is met, secondary sanitization may be deactivated at a step 336, such as by deactivating one or more secondary emitters. Secondary sanitization may thereafter remain deactivated until emission activity is reset as described above.

As can be seen, primary sanitization may remain constantly active while secondary sanitization occurs on a controlled basis. This conserves power utilization and, since secondary sanitization occurs when an area is unoccupied, further ensures that humans are not exposed to or overly exposed to UVC radiation of secondary emitters.

Though described herein with reference to primary and secondary sanitization, it is contemplated that tertiary or other additional sanitization may be provided by the ultraviolet sanitization fixture disclosed herein. Typically, each category of sanitization will have distinct emitters associated therewith that emit distinct wavelengths of UVC radiation.

Referring to FIG. 4, at a step 404 it has been determined that an area is occupied, such as by one or more sensors. At a decision step 408 it may be determined whether primary sanitization should be activated. As described above with regard to steps 308 and 312, primary sanitization will typically be activated on a constant basis. This will typically be the case when one or more humans are present as well. Accordingly, decision step 408 need not be provided in all embodiments and primary sanitization may be activated at a step 412 on a constant basis even when an area is occupied.

As can be seen, secondary sanitization will typically be deactivated when humans are present. This is illustrated at a step 416, where secondary sanitation is deactivated. This prevents over exposure of humans to the associated UVC radiation. It is noted that the recording of emission activity allows sanitization to be interrupted by a human presence and the resumed once the area is unoccupied again. Since the emission activity is recorded, the total emission time or other threshold measure would not exceed the ultraviolet sanitization fixture's associated emission threshold.

FIG. 5 is a block diagram illustrating an exemplary controller 116 of an ultraviolet sanitization fixture. As can be seen, a controller 116 may comprise one or more processors 504. A processor 504 may be a microprocessor, microcontroller, ASIC, FPGA, CPU, or the like. A processor 504 may execute instructions to provide the functionality disclosed herein. Such instructions may be integrated into the circuitry or memory of a processor or may be stored on another non-transient storage device, such as an external storage device 512.

Some exemplary storage devices 512 include flash drives, magnetic drive, optical drives, and the like. A storage device 512 may also store one or more operating parameters, emission thresholds, emission activity, and other data used during operation of an ultraviolet sanitization fixture. A memory device 508 may be provided for temporary storage, such as in the form of RAM or cache memory.

One or more I/O devices 520 may communicate with one or more sensors of an ultraviolet sanitization features, such as to receive sensor information therefrom. In addition, an I/O device 520 may control one or more emitters, such as to activate or deactivate the same. An I/O device 520 may also control power levels at one or more emitters as well.

I/O devices 520 may be provided to communicate with human input devices, such as for programming, configuration, and maintenance of a controller. For example, emission thresholds, emission activity reset triggers, time, passwords and other features may be setup via one or more human input devices. Some exemplary human input devices include keyboards, mice, touch screens, and trackpads. In addition, an I/O device 520 may communicate with various output devices, such as displays, printers, and other output peripherals.

A timing device 516, such as a clock or timer, may be provided for timing purposes. As described above, a timing device 516 may be used to record elapsed activation time. In addition, a timing device 516 may be used to determine when to reset an emission threshold as described above.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. An ultraviolet sanitization fixture comprising:
one or more primary emitters that emit UVC radiation within a first wavelength range, wherein the one or more primary emitters are activated on a substantially constant basis;
one or more secondary emitters that emit UVC radiation with a second wavelength range, the second wavelength range distinct from the first wavelength range; and
a controller that:
records emission activity of the one or more secondary emitters;
activates or deactivates the one or more secondary emitters based on one or more thresholds relative to the recorded emission activity; and
resets the recorded emission activity based on one or more predefined reset triggers.

2. The ultraviolet sanitization fixture of claim 1, wherein the first wavelength range is between 207 nm and 222 nm.

3. The ultraviolet sanitization fixture of claim 1, wherein the second wavelength range is between 254 nm and 265 nm.

4. The ultraviolet sanitization fixture of claim 3, wherein the first wavelength range does not overlap the second wavelength range.

5. The ultraviolet sanitization fixture of claim 1, wherein the emission activity comprises emission time or power output.

6. The ultraviolet sanitization fixture of claim 1, wherein the one or more predefined reset triggers comprise a time limit or detection of a human presence.

7. The ultraviolet sanitization fixture of claim 1, further comprising one or more sensors, wherein the controller deactivates the one or more secondary emitters when a human presence is detected by the one or more sensors.

8. An ultraviolet sanitization fixture comprising:
one or more primary emitters that emit UVC radiation within a first wavelength range;
one or more secondary emitters that emit UVC radiation with a second wavelength range, the second wavelength range distinct from the first wavelength range; and
a controller that:
records emission activity of the one or more secondary emitters; and
activates or deactivates the one or more secondary emitters based on one or more limits relative to the recorded emission activity;
wherein the one or more primary emitters are activated regardless of whether the one or more secondary emitters are activated or deactivated.

9. The ultraviolet sanitization fixture of claim 8, wherein the controller resets the recorded emission activity based on an elapsed time.

10. The ultraviolet sanitization fixture of claim 8, further comprising one or more sensors, wherein the controller resets the recorded emission activity based on detection of a human presence by the one or more sensors.

11. The ultraviolet sanitization fixture of claim 8, further comprising one or more sensors, wherein the controller resets the recorded emission activity based on one or more temperature thresholds measured by the one or more sensors.

12. The ultraviolet sanitization fixture of claim 8, wherein the first wavelength range is between 207 nm and 222 nm.

13. The ultraviolet sanitization fixture of claim 8, wherein the second wavelength range is between 254 nm and 265 nm.

14. The ultraviolet sanitization fixture of claim 8, wherein the emission activity comprises emission time or power output.

15. The ultraviolet sanitization fixture of claim 8, wherein the one or more primary emitters are activated on a substantially constant basis.

16. A method of ultraviolet sanitization with an ultraviolet sanitization fixture comprising one or more primary emitters and one or more secondary emitters, the method comprising:
activating the one or more primary emitters, wherein the one or more primary emitters emit UVC radiation within a first wavelength range;
determining emission activity of the one or more secondary emitters with a controller starting from an occurrence of one or more triggering events; and
activating or deactivating the one or more secondary emitters based on one or more limits relative to the emission activity, wherein the one or more secondary emitters emit UVC radiation with a second wavelength range distinct from the first wavelength range;
wherein the one or more primary emitters are activated regardless of whether the one or more secondary emitters are activated or deactivated.

17. The method of claim 16, wherein the one or more triggering events comprise a predefined period of time.

18. The method of claim 16, wherein the one or more triggering events comprise detection of a human presence by one or more sensors.

19. The method of claim 16, wherein the one or more triggering events comprise detection of one or more particular temperatures by one or more sensors.

20. The method of claim 16, wherein the emission activity comprises emission time or power output.
